(19) United States
(12) Patent Application Publication
(10) Pub. No.: US 2011/0104051 A1
(43) Pub. Date: May 5, 2011

(54) Chemically Modified Viral Capsids as Targeted Delivery Vectors for Diagnostic and Therapeutic Agents

(75) Inventors: Matthew B. Francis, Berkeley, CA (US); Jacob M. Hooker, Charlestown, MA (US); Ernest W. Kovacs, Albany, NY (US); Dante W. Romanini, New York, NY (US); Patrick G. Holder, Somerville, MA (US); Katherine E. Berry, Berkeley, CA (US)

(73) Assignee: THE REGENTS OF THE UNIVERSITY OF CALIFORNIA, Oakland, CA (US)

(21) Appl. No.: 12/294,743

(22) PCT Filed: Mar. 27, 2007
(86) PCT No.: PCT/US2007/007484

§ 371 Date: Jan. 5, 2011

Related U.S. Application Data
(60) Provisional application No. 60/785,979, filed on Mar. 27, 2006.
(60) Provisional application No. 60/840,040, filed on Aug. 25, 2006.

(51) Int. Cl. A61K 51/04; A61K 9/00; A61K 49/00; A61P 35/00; A61K 47/42

(52) U.S. Cl. 424/1.17; 424/400; 424/9.1; 514/773

(57) ABSTRACT

The present invention provides a novel means of delivering imaging agents and/or therapeutic agents to specific cellular sites in an animal involving the use of a viral capsid that is chemically modified on it exterior and/or interior surfaces.

### RELATED APPLICATIONS

This application claims the benefit of priority of U.S. Provisional Applications 60/840,040, filed Aug. 25, 2006 and 60/785,979, filed Mar. 27, 2006, the disclosures of which are incorporated herein by reference for all purposes.

### ACKNOWLEDGEMENT OF GOVERNMENT SUPPORT

This invention was made in part with government support under Grant No. NIH GM072700-01 awarded by the National Institutes of Health (NIH) and Grant No. DMR 9808677 awarded by the National Science Foundation (NSF). The government may have certain rights in this invention.

### BACKGROUND OF THE INVENTION

The beneficial effects of pharmaceutical compounds generally arise from their interactions with specific cellular targets. Most current methods of administration, however, nonselectively deliver a therapeutic agent to a patient, thus exposing the agent to virtually all tissue types. Not surprisingly, this approach often results in increased side effects and toxicity experienced by the patient, and can be particularly devastating in the case of potent therapeutic agents, such as anti-cancer compounds. As a result, much attention has been directed toward the generation of carrier systems that are able to deliver therapeutic agents selectively to the desired cellular targets. See, e.g., Kopecek et al., Eur. J. Pharm. Biopharm. 50, 61-81 (2000); Duncan, Cancer Res. 46, 175-210 (1992); Maeda et al., Bioconjugate Chem. 3, 351-362 (1992).

In addition to reducing deleterious side effects, it is envisaged that such carrier systems could also protect therapeutic agents from premature degradation or excretion, thus further increasing agent efficacy and lowering agent dosage levels. These carrier systems could also be used to selectively deliver a variety of agents, such as metal complexes and radioisotopes, to desired tissue types, thereby generating accurate and sensitive imaging techniques (e.g., positron emission tomography (PET) and magnetic resonance imaging (MRI)) for diagnostic applications. These potential benefits suggest that the development of effective and targetable delivery vectors could provide a quantum leap in disease diagnosis and treatment.

To date, several approaches to improved drug delivery have been explored, including the use of liposomes and synthetic polymers. See, e.g., Sharma et al., Int. Jour. Pharm. 154(2), 123 (1997); Kaneda et al., Advanced Drug Delivery Reviews 43, 197-205 (2000) and Duncan, Controlled Drug Delivery, ACS Symposium Series 752, Chapter 33, 350-363 (2000).

The robust, monodisperse architectures of viral capsids have emerged as attractive scaffolds for the construction of new materials. Viruses are natural carriers that are uniquely capable of protecting and selectively delivering their genetic contents to cells and are sufficiently large to possess increased plasma residence times. As such, viruses have been considered in the context of gene delivery as a means of delivering beneficial DNA into cells instead of the viruses' native genetic information. See, e.g., Marshall, Science 288, 953 (2000); Hackett et al., Curr. Opin. Mol. Ther. 2, 376-382 (2000). With the goal of adding new function to these structures, several studies have demonstrated that their exterior surfaces can be functionalized with peptides, fluorescent dyes, polymers, carbohydrates, oligonucleotides, and other organic molecules through the use of carefully planned bioconjugation reactions.

In terms of biomedical applications, the construction of spherical nanomaterials with differentiated surfaces would provide particularly attractive systems for the delivery of therapeutic agents. As suggested in FIG. 1, an ideal construct for these purposes would consist of an externally modified surface presenting multiple copies of a ligand that targets specific cell receptors, while also providing resistance to antibody binding that would neutralize the carriers before reaching their destinations. Interior modification strategies could then be used to install a high payload of imaging agents, drug cargo, or other molecules of biomedical interest in a modular fashion, with the advantage that internal functionality would be expected to exert minimal effects on biodistribution. Even in the absence of specific targeting with ligands, passive accumulation of these materials could occur in solid tumors by way of the enhanced permeability and retention (EPR) effect. Though we have previously demonstrated the dual surface covalent modification of the tobacco mosaic virus (TMV) with small molecules and polymers, there remain no examples of icosahedral capsids that have been simultaneously modified on both their exterior and interior surfaces using orthogonal chemical reactions.

In terms of inorganic materials, the capsids have also served as templates for the positioning of gold nanoparticles, the deposition of metal oxides, and the coordination of gadolinium ions for magnetic resonance imaging (MRI). Similarly, the interior surfaces of some viral capsids have been exploited as “cages” that can template the growth of inorganic nanocrystals and display organic functionality. Beyond these synthetic methods, much attention has also been devoted to the genetic manipulation of capsid proteins to present peptide sequences with desired function. Specifically, capsid proteins have been expressed as fusion proteins to display antigenic peptides on their surfaces, and the evolution of peptide sequences via phase display has achieved promising and widely applicable success for a number of applications.

A major disadvantage of this approach is the rapid inactivation of the altered viruses by the host's immune system, which stands as one of the major obstacles in gene therapy. The present invention overcomes this immunogenicity problem by chemically modifying the exterior surface of a viral capsid with at least one polymer such that the capsid is effectively shielded from an immune response by the recipient of the capsid. For example, poly(ethylene glycol) (PEG) chains are known to inhibit the recognition of proteins by immunoglobulins. See e.g., Harris et al, eds., Poly(ethylene glycol): Chemistry and Biological Applications, American Chemical Society (1997). The exterior and/or interior surfaces of the capsid may also be chemically modified to attach imaging agents for diagnostic applications or therapeutic agents for disease treatment.

In the present invention, genome-free capsids have been selectively modified on their exterior and/or interior surfaces. Utilizing various coupling strategies, polymer chains have been successfully appended to the exterior surface of selected capsids while decorating their interior surfaces with any of several drug or drug mimics. This dual-surface modification has been achieved in only two protein modification steps with high overall recovery. In vitro ELISA assays, furthermore, have shown that the extensive polymer chain formation (e.g., PEGylation) achieved in the present invention effectively masks the epitopes of the native capsid surface. A modular strategy has also been developed to attach targeting groups to the distal ends of the polymer chains through chemoselective oxime formation reactions. These constructs represent a synthetic exploitation of the three-dimensional space afforded by the capsids, and provide a promising platform for future biomedical applications. This modification of the exterior and/or interior surface of a capsid allows for customization of the capsid's immunogenicity, solubility, stability and targeting properties to suit the particular envisaged application.

### SUMMARY OF THE INVENTION

The present invention provides a novel means of delivering imaging agents and/or therapeutic agents to specific cellular sites in an animal involving the use of a viral capsid that is chemically modified on it exterior and/or interior surfaces.

An aspect of the invention is a method of using a viral capsid comprising an interior surface and an exterior surface to deliver a therapeutic agent to a selected cell of a mammal in need thereof, comprising modifying the exterior surface by covalently attaching a polymer, removing the capsid's native genome, and modifying the interior surface by covalently attaching a therapeutic agent, wherein the covalent attachment is cleaved by conditions present in the cell, and administering the resulting modified capsid to the mammal.

Another aspect of the invention is a method of using a viral capsid comprising an interior surface and an exterior surface to deliver a diagnostic imaging agent to a selected cell of a mammal in need thereof, comprising removing the capsid's native genome, modifying at least one of the exterior surface and the interior surface by covalently attaching the diagnostic imaging agent, and administering the resulting modified capsid to the mammal.

Another aspect of the invention is a viral capsid for delivery of a therapeutic agent to selected cells of a mammal in need thereof, comprising an interior surface to which a therapeutic agent is covalently attached; and an exterior surface to which a polymer is covalently attached, wherein the capsid's native genome has been removed.

Another aspect of the invention is a viral capsid for delivery of a therapeutic agent to selected cells of a mammal in need thereof, comprising an exterior surface to which a diagnostic imaging agent is covalently attached, wherein the capsid's native genome has been removed.

Another aspect of the invention is a viral capsid for delivery of a therapeutic agent to selected cells of a mammal in need thereof, comprising an interior surface to which a diagnostic imaging agent is covalently attached, wherein the capsid's native genome has been removed.

Other objects and advantages of the invention will be apparent to those of skill in the art from the detailed description that follows.

### DESCRIPTION OF THE DRAWINGS

The following figures are merely specific embodiments of the present invention and are not intended to otherwise limit the scope of the claimed invention.

FIG. 1 depicts an exemplary embodiment of the present invention, where, in a first step, the exterior surface of a native MS2 virus is chemically modified with a polymer coating. In a second step, the native RNA of the virus is removed, leaving an empty capsid shell with a functionalized exterior. In a third step, the interior of capsid is chemically modified with a therapeutic agent, wherein the therapeutic agent is attached to the interior surface through an acid-cleavable linking group. Upon administration to a recipient, the modified capsid is taken up into a targeted cell via endocytosis. The low pH environment of the cell's endosome and/or lysosome cleaves the therapeutic agent from the interior surface of the capsid, releasing the therapeutic agent inside the cell.

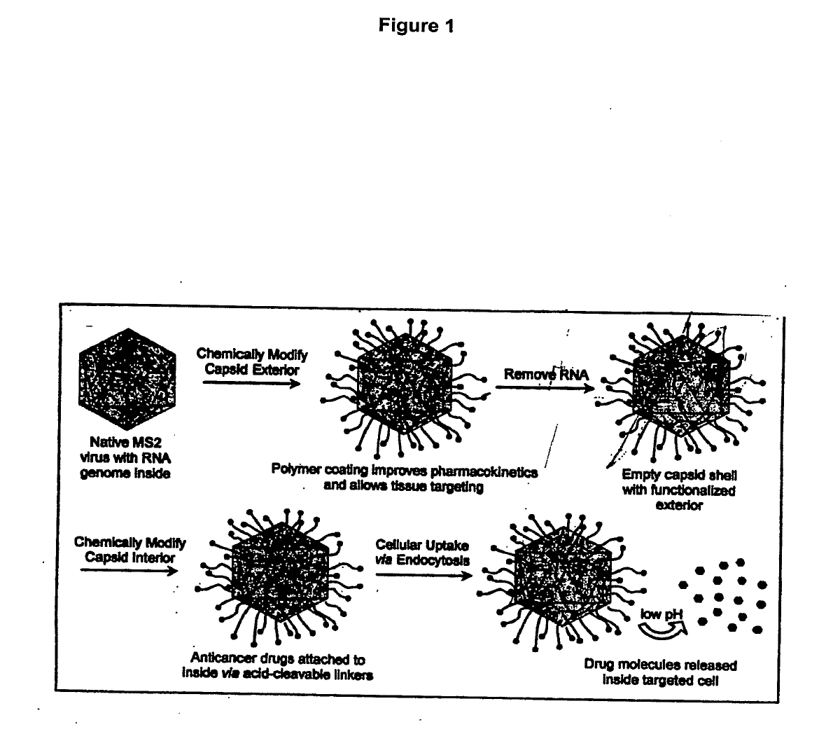

FIG. 2 depicts an exemplary embodiment of the present invention in which the exterior surface of a MS2 virus lacking its native genome is chemically modified through a cysteine residue with a poly(ethylene glycol)alkoxyamine of MW 2000 or 5000; and the interior surface of a MS2 virus lacking its native genome is chemically modified through a tyrosine residue.

FIG. 3 depicts an exemplary embodiment of the present invention showing dual surface modification of MS2 capsids. In (a), tyrosine 85 of the interior capsid undergoes rapid diazonium coupling with p-nitroaniline derivatives, including large dye conjugates. In (b), up to 360 accessible amino groups (lysines 106, 113 and the N-terminus) on the capsid exterior are readily modified with PEG-NHS esters. In (c), SDS-PAGE analysis confirms both PEGylation (via MW shift) and dye attachment (fluorescent bands) for capsid monomers after disassembly. Lanes A and G: MW markers; lane B: natMS2; lane C: mtMS2 (1)+aniline (5); lane D: (2): lane E: (3a); lane F: (3b). In addition to other methods, the assembly state of capsids (3a) and (3b) was confirmed by TEM analysis after staining with UO₂(OAc)₂.

FIG. 4 depicts an exemplary embodiment of the present invention showing a dual surface modification sequence. Reagents and conditions are as follows: (a) DMF, Et₃N, 65° C., 1.5 h, >95%; (b) NaNO₂, p-TsOH, 0° C., 0.75 h; (c) 6 (5 equiv), pH 8.5, 0° C., 5 min, 90% protein recovery; (d) pH 8.4, rt, 15 h, 90% protein recovery.

FIG. 5 depicts an exemplary embodiment of the present invention showing the results of an evaluation of PEG-MS2 binding by polyclonal antibodies using an enzyme-linked immunosorbent assay (ELISA). Virtually no binding of the modified capsids could be discerned above the background levels measured using a control protein (BSA). This data indicates that the PEG-modified capsids remain undetected during transport to their tissue targets.

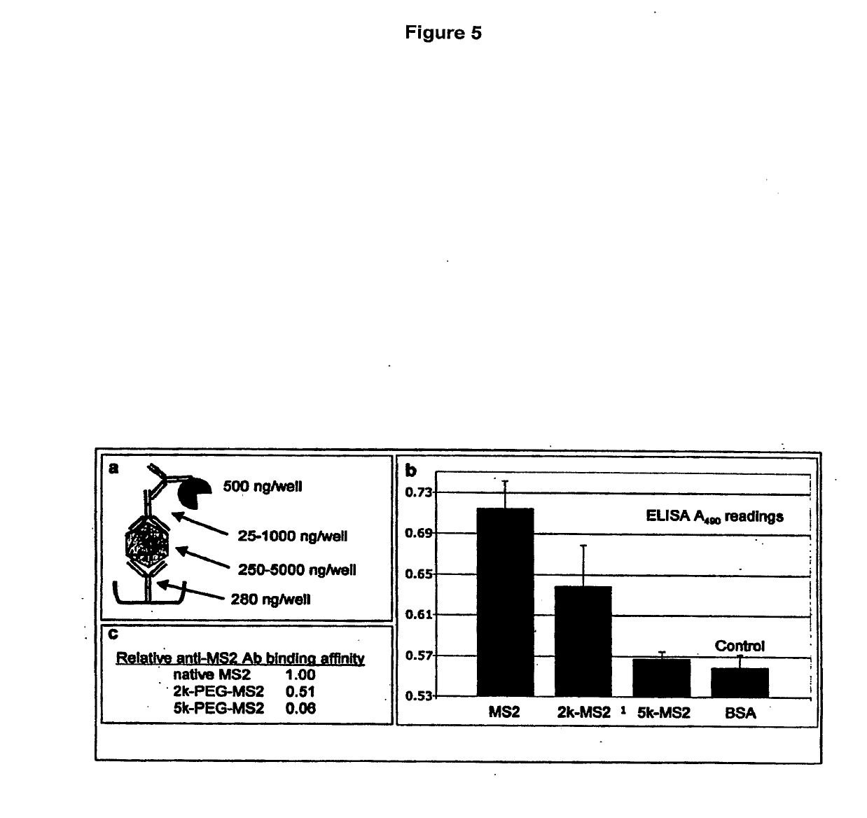

FIG. 6 depicts an exemplary embodiment of the present invention showing a polyclonal anti-natMS2 antibody response to MS2 capsids, as determined by ELISA. (a) ELISA response for the sample antigen concentration from 10 ng/mL to 0.1 mg/mL. (b) Comparative antibody binding for antigen concentration at 0.1 mg/mL. BSA=bovine serum albumin and chymo=α-chymotrypsinogen (negative controls).

FIG. 7 depicts an exemplary embodiment of the present invention showing accessibility of small molecules displayed on the distal ends of MS2-PEG conjugates. (a) Biotinylated samples of MS2 were prepared through reaction of the corresponding NHS esters with genome-free capsids. Densitometry measurements after SDS-PAGE analysis and Coomassie staining indicated that approximately 30% of the monomers were labeled in 8a and approximately 70% of the monomers were labeled in 8b. (b) Conjugates 7, 8a, and 8b were incubated with avidin beads for 0, 1 and 15 h. (c) Following centrifugation, the MS2 remaining in the supernatant was analyzed by SDS-PAGE. The percentages reported were determined by densitometry analysis after Coomassie staining. For the polymer-conjugated samples, only the band corresponding to unlabeled monomers is shown.

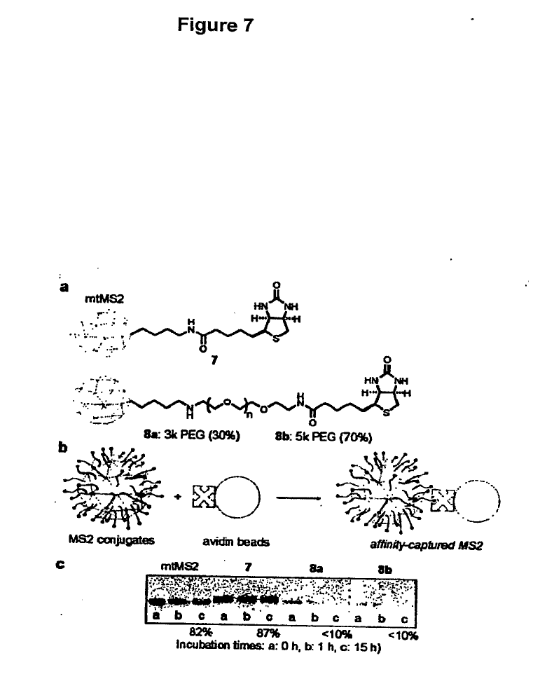

FIG. 8 depicts an exemplary embodiment of the present invention showing a chemical strategy for the installation of PEG chains bearing small molecule targeting groups. Reagents and conditions: (a) DIAD, N-hydroxyphthalimide, CH₂Cl₂, rt, 12 h; (b) NH₂NH₂.xH₂O (2.2 equiv.), CH₂Cl₂, rt, 1 h; (c) CH₂Cl₂, 0.1% TFA; (d) 100 mM NaH₂PO₄ aqueous buffer, pH 9.0, rt, 2 h; (e) 25 mM NaH₂PO₄ aqueous buffer, pH 6.5, 1.5 h, rt.

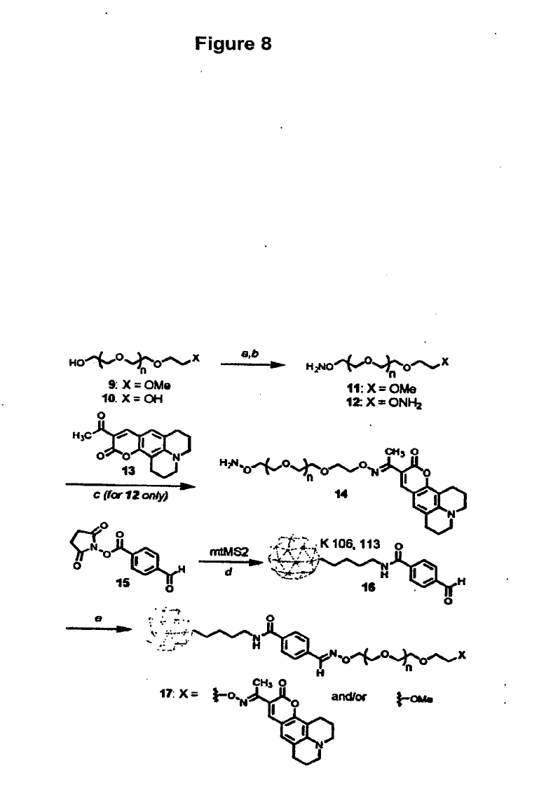

FIG. 9 depicts an exemplary embodiment of the present invention showing attachment of functionalized PEG chains to the aldehyde groups of MS2 conjugate 16 through oxime formation. All reactions were carried out in 25 mM phosphate buffer, pH 6.5 for 1.5 h using the indicated ratios of 14 and 11. The remaining polymer was removed via gel filtration before SDS-PAGE analysis. From top to bottom, the MW markers correspond to 15, 20 and 25 kD.

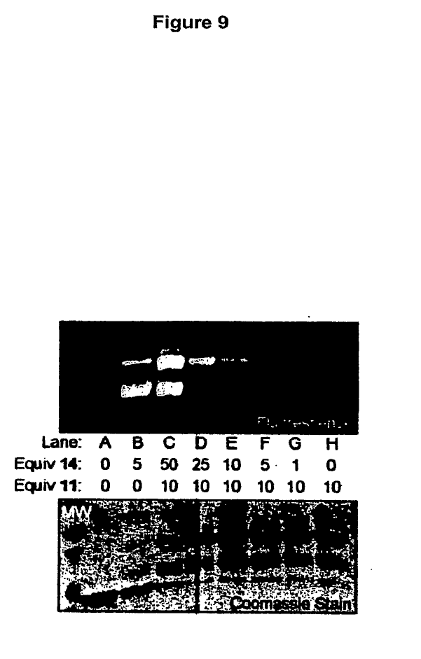

FIG. 10 depicts an exemplary embodiment of the present invention in which 1,4,7,10-tetraazacyclododecane-1,4,7,10-tetraacetic acid (DOTA)-NHS is used to modify available lysine residues on the exterior of the capsid shell. As shown by the graphs, a significant percentage (approximately 55%) of the lysines are converted to the DOTA conjugates. Once the conjugate was formed, metal ions such as Gd(³⁺), Cu(²⁺), Tb(³⁺), Yb(³⁺) and Eu(³⁺) were observed to bind to the capsid in near quantitative yield.

### DETAILED DESCRIPTION OF THE INVENTION

### Definitions

Unless defined otherwise, all technical and scientific terms used herein generally have the same meaning as commonly understood by one of ordinary skill in the art to which this invention belongs.

As defined herein, “immune response” refers to a humoral immune response and/or cellular immune response leading to the activation or proliferation of B- and/or T-lymphocytes and/or and antigen presenting cells.

As defined herein, “amino acid residue” or “residue” refers to a specific amino acid in a polypeptide backbone or side chain.

As defined herein, “viral capsid” refers to the shell of protein that protects the nucleic acid of a virus. The viral capsid may be chemically modified by covalently attaching chemical moieties, such as polymer chains, imaging agents and therapeutic agents, to the interior and/or exterior surfaces of the capsid.

As defined herein, “therapeutic agent” refers to any agent useful for therapy including, but not limited to, antibiotics, anti-inflammatory agents, anti-tumor drugs, cytotoxins and radioactive agents. “Therapeutic agent” includes prodrugs of bioactive agents and constructs in which more than one therapeutic agent is bound to a carrier, e.g., multivalent agents. “Therapeutic agent” also includes proteins and constructs that include proteins.

As defined herein, “vector” refers to a carrier or vehicle, such as, for example, a viral capsid, for the transmission of a substance from one site to another site.

As defined herein, “targeted delivery” refers to the localized deposit of a substance to a particular tissue or cell type. The localization is mediated by specific recognition of molecular determinants, molecular size, ionic interactions, hydrophobic interactions and the like. Additional mechanisms of delivering a substance to a particular tissue or cell type or region of the body are known to those of skill in the art.

As defined herein, herein, “pharmaceutically acceptable carrier” includes any material, which when combined with a chemically modified viral capsid retains the capsid's activity and is non-reactive with the recipient's immune systems. Examples include, but are not limited to, any of the standard pharmaceutical carriers such as a phosphate buffered saline solution, water, emulsions such as oil/water emulsion, and various types of wetting agents. Compositions comprising such carriers are formulated by well known conventional methods.

As defined herein, “administering” refers to any of oral administration, administration as a suppository, topical contact, intravenous, intraperitoneal, intramuscular, intralesional, intranasal or subcutaneous administration, or the implantation of a slow-release device (e.g., a mini-osmotic pump) in the subject. Administration may occur by any route, including parenteral and transmucosal (e.g., oral, nasal, vaginal, rectal, or transdermal). Parenteral administration includes, for example, intravenous, intramuscular, intra-arteriole, intradermal, subcutaneous, intraperitoneal, intraventricular, and intracranial. Moreover, where the injection is to treat a tumor (e.g., to induce apoptosis), administration may be directly to the tumor and/or into tissues surrounding the tumor. Other modes of delivery include, but are not limited to, the use of liposomal formulations, intravenous infusion, transdermal patches, etc.

As defined herein, “therapy” refers to the treating or treatment of a disease or condition that includes preventing the disease or condition from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

As defined herein, “effective amount” or “an amount effective to” or a “therapeutically effective amount” or any grammatically equivalent term refers to the amount that, when administered to an animal for treating a disease or condition, is sufficient to effect treatment for that disease or condition.

As defined herein, the term “alkyl,” by itself or as part of another substituent refers to, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

As defined herein, the term “alkylene” by itself or as part of another substituent refers to a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

As defined herein, the terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

As defined herein, the term “heteroalkyl,” by itself or in combination with another term, refers to, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

As defined herein, the terms “cycloalkyl” and “heterocycloalkyl”, by themselves or in combination with other terms, refer to, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

As defined herein, the terms “halo” or “halogen,” by themselves or as part of another substituent, refer to, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

As defined herein, the term “aryl” refers to, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, tetrazolyl, benzo[b]furanyl, benzo[b]thienyl, 2,3-dihydrobenzo[1,4]dioxin-6-yl, benzo[1,3]dioxol-5-yl and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, alkylaryl) includes both aryl and heteroaryl rings as defined above. Thus, as defined herein, the term “alkylaryl” refers to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) is meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present. In the schemes that follow, the symbol X represents “R” as described above.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′ or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As defined herein, the term “heteroatom” refers to include oxygen (O), nitrogen (N), sulfur (S) and silicon (Si).

### Chemical Modification of the Capsid Exterior

The basic strategy for chemical modification of the exterior of the viral capsid targets amino acid residues on the exterior surface that contain free alcohol (—OH), thiol (—SH) or amino (—NH₂ or —NHR, where R is alkyl or acyl) moieties and that are available for reaction with a linking group (L) that connects the amino acid to a synthetic polymer (P) as characterized below. Two requirements of the synthetic polymer are that it is non-toxic and that it prevents the capsid from eliciting an immune response when administered to a recipient.

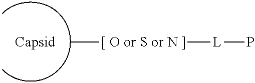

In exemplary embodiments, the reacting amino acid contains a thiol, alcohol or amino moiety. In a particular embodiment, the thiol moiety is part of a cysteine residue that is present on the exterior surface of the capsid. In another embodiment, the alcohol moiety is part of a serine, tyrosine or threonine residue that is present on the exterior surface of the capsid. In a particular embodiment, the alcohol moiety is part of a tyrosine residue. In yet another embodiment, the amino moiety is part of a lysine or arginine residue this present on the exterior surface of the capsid. In a particular embodiment, the amino moiety is part of a lysine residue.

Examples of suitable virus capsids include, but are not limited to, capsids from sindbis and other alphaviruses, rhabdoviruses (e.g. vesicular stomatitis virus), picornaviruses (e.g., human rhino virus, Aichi virus), togaviruses (e.g., rubella virus), orthomyxoviruses (e.g., Thogoto virus, Batken virus, fowl plague virus), polyomaviruses (e.g., polyomavirus BK, polyomavirus JC, avian polyomavirus BFDV), parvoviruses, rotaviruses, bacteriophage Qβ, bacteriophage R17, bacteriophage M11, bacteriophage MX1, bacteriophage NL95, bacteriophage fr, bacteriophage GA, bacteriophage SP, bacteriophage MS2, bacteriophage f2, bacteriophage PP7, bacteriophage AP205, Norwalk virus, foot and mouth disease virus, a retrovirus, Hepatitis B virus, Tobacco mosaic virus (TMV), satellite panicum mosaic virus (SPMV), Flock House Virus, and human Papilomavirus.

In an exemplary embodiment, the viral capsid is that of the bacteriophage MS2. The MS2 virus comprises a single strand of RNA (approximately 3,600 nucleotides) encased in a capsid that is approximately 27 nm in diameter and which is assembled from multiple copies of a single protein. MS2 infects specific strains of E. coli as its host and is harmless to mammals, including humans. MS2 has the advantage of being easily propagated using routine broth culture techniques and is purified using a precipitation procedure. See, e.g., Davis et al., J. Mol. Biol. 6, 203-207 (1963). Typical yields of MS2 from this procedure are about 30 mg of highly pure virus per liter of broth. In more recent studies, the direct expression of the MS2 coat protein in E. coli culture has also yielded substantial quantities of assembled capsids.

In another exemplary embodiment, the viral capsid is that of TMV.

The linking group (L) is not particularly limited and may be a direct bond or any of a number of chemical moieties that allow covalent attachment of the polymer to a suitable amino acid residue on the exterior surface. Such chemical moieties may include, for example, an alkyl group, an aryl group, an alkylaryl group or a diazo group. In an exemplary embodiment, the linking group is a maleimide derivative of the following formula

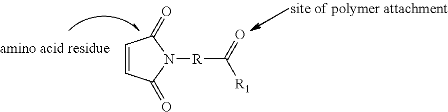

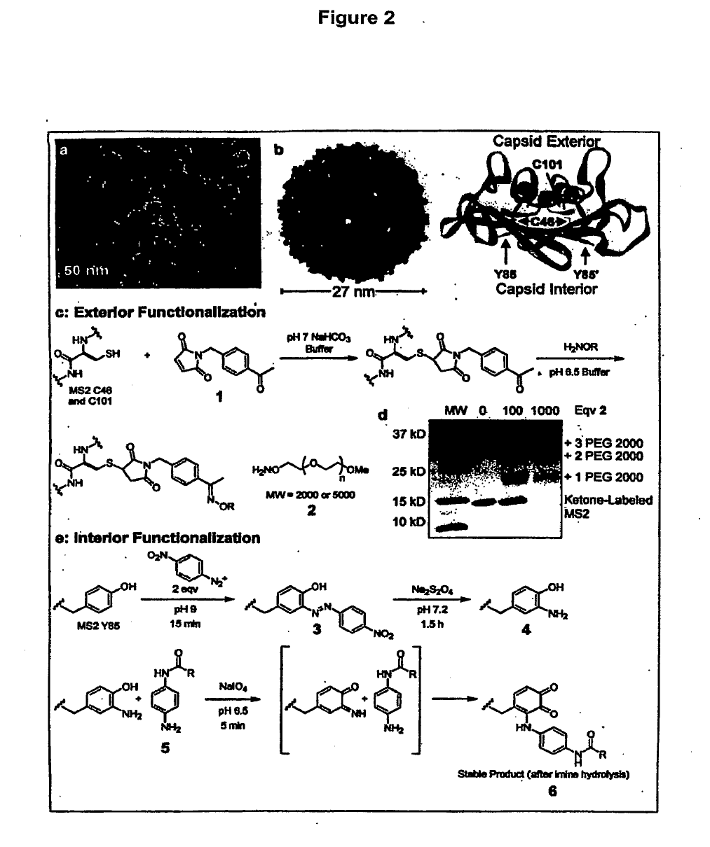

as shown in FIG. 2. In one embodiment, R is —CH₂-Ph- and R₁ is —CH₃. In another exemplary embodiment, the linking group is a succinimide. In a particular embodiment, the succinimide is a N-hydroxysuccinimidyl(4-acetyl)benzoate. The manner in which the polymer may be attached to the linking group is not particularly limited. In an exemplary embodiment, an alkoxyamine derivative of the polymer reacts with a carbonyl moiety on the linking group to form the corresponding oxime as shown in FIG. 2. In an exemplary embodiment, the alkoxyamine is a poly(ethylene glycol)alkoxyamine. In another embodiment, the poly(ethylene glycol)alkoxyamine is a O-(methoxypolyethylene glycol)alkoxyamine.

The polymer is also not particularly limited. In an exemplary embodiment, the polymer is a poly(alkylene oxide). Poly(alkylene oxide) refers to a genus of compounds having a polyether backbone. Poly(alkylene oxide) species of use in the present invention include, for example, straight- and branched-chain species. Moreover, exemplary poly(alkylene oxide) species can terminate in one or more reactive, activatable, or inert groups. For example, poly(ethylene glycol) (PEG) is a poly(alkylene oxide) consisting of repeating ethylene oxide subunits, which may or may not include additional reactive, activatable or inert moieties at either terminus. Useful poly(alkylene oxide) species include those in which one terminus is “capped” by an inert group, e.g., monomethoxy-poly(alkylene oxide). When the molecule is a branched species, it may include multiple reactive, activatable or inert groups at the termini of the alkylene oxide chains and the reactive groups may be either the same or different. poly(ethylene glycol). A suitable range for the molecular weight of an individual PEG chain is between about 500 to about 50,000 daltons, such as, for example, between about 1,000 and about 25,000, or between about 1,500 and about 15,000. In an exemplary embodiment, an individual PEG chain has a molecular weight of about 2,000 daltons (PEG-2000). In another embodiment, an individual PEG chain has a molecular weight of about 5,000 daltons (PEG-5000).

Typically, the amino acid residues present on the exterior surface of the capsid that are suitable for linking with the synthetic polymers of the present invention (i.e., those amino acids containing reactive alcohol, thiol or amino groups) are modified with high efficiency despite the steric crowding that the polymer chains may impose. For example, using MS2 as an exemplary viral capsid and PEG-2000 as an exemplary polymer, experiments demonstrated that an estimated 650,000 daltons of total polymer was added to the capsid shell. When the polymer is PEG-5000, experiments indicated that an estimated 1.5 MDa of total polymer was added to the capsid shell. This technique has also been used to install small molecules, including folic acid, to the distal ends of polymer chains to target the structure to solid tumors and other tissues of clinical interest. See e.g., Sudimack et al., Adv. Drug Delivery Rev. 147 (2000).

### Preparation of Genome-Free Viral Capsids

Before the viral capsids can be used as targeted delivery vectors for diagnostic and/or therapeutic agents, the native genome of the virus must be removed. In an exemplary embodiment, the viral capsid contains a plurality of pores through which the genomic material may be removed and small molecules representing, for example, linking groups, therapeutic and/or imaging agents may enter. In another exemplary embodiment, the native genome is RNA.

Typically, the virus is exposed to alkaline conditions which results in degradation of the genome and escape of the cleaved nucleotides through the pores of the capsid. In an exemplary embodiment, the virus is exposed to pH conditions of about 8 to about 14 for a period of time of about 15 minutes to about 10 hours. In another embodiment, the virus is exposed to pH conditions of about 9 to about 13 for about 30 minutes to about 6 hours. In another embodiment, the virus is exposed to pH conditions of about 10 to about 12 for about 1 to about 4 hours. In yet another embodiment, the virus is exposed to pH conditions of about 11 to about 12 for about 2 to about 4 hours. Following this procedure, the now “empty” capsids (i.e., devoid of native genetic material) can be isolated through, for example, precipitation techniques, with greater than about 80% overall protein recovery. The empty capsids are stable in the pH range of about 3 to about 9 over a 12 hour period, with only minor losses occurring at a pH of less than about 3 or greater than about 10. Further, the empty capsids are stable at temperatures as high as about 60° C.

Empty capsids can be readily distinguished from capsids containing native genomic material based on any of several known techniques. For example, empty capsids appear dark when exposed to a UO₂(OAc)₂ stain, in contrast to capsids that still possess genomic material. UV spectral analysis of empty MS2 capsid shells isolated using, for example, gel filtration indicates that the characteristic RNA absorbance at 260 nm is absent.

### Modification of the Viral Capsid Interior

Following removal of the viral genome, the interior surface of the capsid may be chemically modified to covalently attach, for example, a therapeutic or an imaging agent. In an exemplary embodiment, the modifying of the interior surface comprises treating an amino acid residue that is affixed to the interior surface of the capsid with the therapeutic agent under conditions sufficient to covalently attach the therapeutic agent directly or indirectly to the amino acid residue. In an exemplary embodiment, the therapeutic agent is an anticancer agent.

The strategy for chemically modifying the interior surface may be similar to the strategy used for chemical modification of the exterior surface of the capsid, where amino acid residues on the interior surface that contain free alcohol or thiol or amino moieties are targeted for reaction with a linking group (L) that connects the amino acid to a therapeutic agent (T) as characterized below.

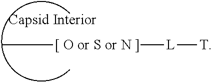

In exemplary embodiments of the invention, the amino acid residue is selected from the group consisting of cysteine, serine, tyrosine, lysine and arginine.

The linking group (L) may be any moiety such that the bond between L and T is cleavable under the acidic conditions generally present inside a cell's endosome and/or lysosome, thus releasing the therapeutic agent inside the targeted cell. Typically, the endosomal environment is at a pH of about 4 to about 6, such as about 4.5 to about 5.5, such as about 5.5, while the lysosomal environment is at a pH of about 4 to about 5, such as about 4.5.

In an exemplary embodiment of modification of an amino acid residue on the interior surface of a suitable viral capsid, a tyrosine residue serves as a linking group between a therapeutic agent and the interior surface. MS2, as an exemplary viral capsid, is known to contain about 180 exposed tyrosine residues on the interior surface that are suitable for modification. As shown in FIG. 2, which depicts this exemplary embodiment, one of these available tyrosine residues is treated with an aromatic diazonium salt to provide the diazo derivative 3. Reduction with a representative reducing agent, such as, for example, sodium dithionite, provides the amino-substituted tyrosine derivative 4. Derivative 4 is then oxidized to an ortho-iminoquinone with an exemplary oxidizing agent such as sodium periodate. The ortho-iminoquinone is a highly reactive species that is able to couple with a wide variety of nucleophiles, such as the phenylene diamine derivative shown. The resulting product 6 is a stable species that covalently links associated functionality to the capsid interior.

The progress of the reaction to form the tethered therapeutic agent may be monitored using, for example, mass spectrometry (e.g., MALDI-TOF). For the particular product 6, in FIG. 2, mass spectrometry indicates that greater than about 60% of the available sites on the interior surface of the viral capsid have been modified. In another embodiment, greater than about 80% of the available sites on the interior surface of the viral capsid have been modified. In yet another embodiment, greater than about 95% of the available sites on the interior surface of the viral capsid have been modified. These modified capsids are easily purified from small molecule reactants through chromatographic means, such as size exclusion chromatography. Once the modified capsids are administered to a recipient, they are directed to a preselected type of cell or tissue where they experience cellular uptake via endocytosis. The modified capsids then encounter the cell's lysosome and endosome, where their resulting acidic environments cleave the therapeutic agent from the modified tyrosine derivative, thereby releasing the therapeutic agent into the cell.

### Installation of Diagnostic Imaging Agents

In an exemplary embodiment, the viral capsid is modified by treating an amino acid residue that is affixed to the at least one exterior or interior surface of the capsid with a diagnostic imaging agent under conditions sufficient to covalently attach the diagnostic imaging agent directly or indirectly to the amino acid residue. In an exemplary embodiment, the diagnostic imaging agent is attached to the amino acid residue through a linking agent.

The modification strategies of the invention may also be used to attach a series of metal complexes to the viral capsid surface for diagnostic imaging purposes. Viral capsids labeled with imaging agents are effective in locating areas of interest within a recipient's body, such as areas of inflammation or tumor metastasis in a patient suspected of having an inflammation. In an exemplary embodiment, a metal binding ligand is covalently attached to suitable amino acid residues present on capsid shell (e.g., those amino acids containing thiol or alcohol or amino moieties, such as, for example, cysteine, serine, tyrosine, lysine and arginine). In an exemplary embodiment, the amino acid residue is a lysine.

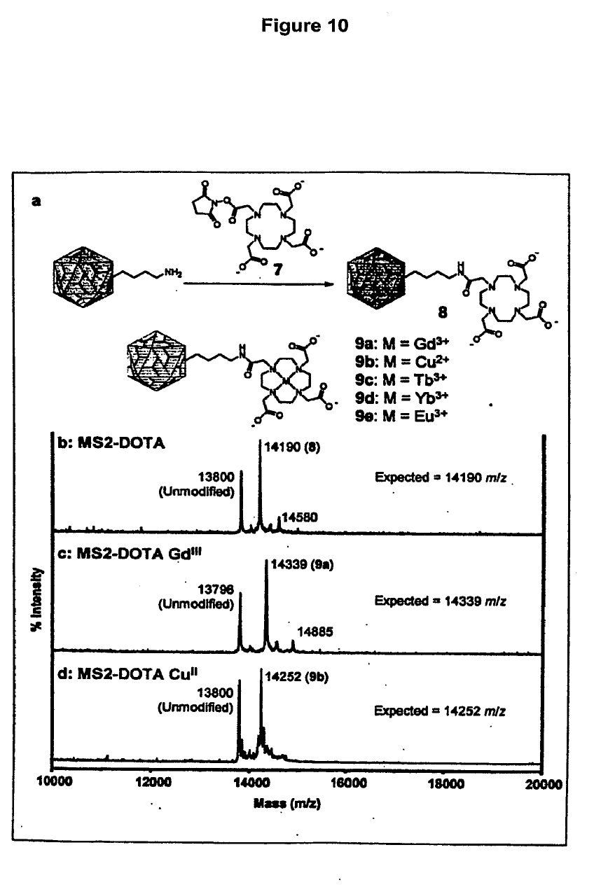

In a particular embodiment, as shown in FIG. 10, the metal binding ligand is 1,4,7,10-tetraazacyclododecane-1,4,7,10-tetraacetic acid (DOTA)-NHS and the amino acid residue through which the DOTA-NHS is attached to the capsid surface is a lysine. Although this method primarily results in labeling on the exterior surface of the capsid, amino acid residues on the interior surface of the capsid may also be suitable for modification. Labeling of the exterior surface amino acid residues with diagnostic imaging agents does not preclude attachment of the earlier described immunoprotective polymeric chains as long as there are amino acid residues containing thiol, alcohol and/or amino moieties still available for linking. For an exemplary virus such as MS2, up to about 55% of the available lysine residues are converted to the DOTA conjugates, indicating that each capsid contains about 100 copies of the metal binding ligand.

Metal ions generally associated with imaging agents, such as, but not limited to, Gd³⁺, Cu²⁺, Tb³⁺, Yb³⁺ and Eu³⁺, may be reacted with the metal binding ligand in nearly quantitative yield to form a complex of the metal with the ligand. In a particular embodiment, the metal ion is Gd³⁺. In another exemplary embodiment, Gd³⁺-DOTA represents the combination of a specific metal ion with a specific metal binding ligand. No binding of the metal ion to the capsid shell takes place in the absence of a metal binding ligand such as DOTA.

Metal ions complexed with metal binding ligands have been used for MRI contrast enhancement. As such, these complexes are anticipated to be useful for imaging applications as prepared. The high molecular weight of the assembled capsids should ensure prolonged circulation times and therefore dramatically lower the overall quantities of the metal ions such as Gd³⁺ that must be administered to obtain proper images. Further, the size of the labeled virus capsids should promote their selective accumulation in tumor tissue due to the well known Enhanced Permeability and Retention (EPR) effect. See e.g., Baban et al., Adv. Drug Delivery Reviews 34, 109-119 (1998); Maeda et al., Controlled Release 65, 271-284 (2000). Addition of various targeting ligands on the capsid exterior could also be used to target other tissue types.

In positron emission tomography (PET) imaging, ⁶⁰Cu²⁺ ions would likely be bound indistinguishably from the naturally occurring ⁶³Cu²⁺ and ⁶⁵Cu²⁺ ions that are indicated in FIG. 10d as being bound to the DOTA ligand.

In another exemplary embodiment of diagnostic imaging, especially for PET, the diagnostic imaging agent is a radionuclide such as, for example, oxygen-15 (¹⁵O), nitrogen-13 (¹³N), carbon-11 (¹¹C), iodine-131 (¹³¹I) or fluorine-18 (¹⁸F), or a compound labeled with a radionuclide. Exemplary embodiments of compounds labeled with ¹⁸F for use in, for example, PET include 2-¹⁸F-2-deoxy-D-glucose and various ¹⁸F-radionucleotides. ¹³¹I may also be employed in various nucleotides.

In another embodiment of the invention, the diagnostic imaging agents may be attached to the interior surface of the viral capsid, such as through the earlier discussed tyrosine modification that proceeds through the ortho-iminoquinone.

The following examples are provided to illustrate specific compositions and methods of the present invention, but are not intended to limit the claimed invention.

### EXAMPLES

### General Procedures and Materials

Unless otherwise noted, all chemicals were of analytical grade obtained from commercial sources and used without further purification. All non-aqueous reactions were carried out under a nitrogen atmosphere using distilled solvents. Water (ddH₂O) used in biological procedures or as a reaction solvent was deionized using a NANOpure™ purification system (Barnstead, USA). All organic solvents were removed under reduced pressure using a rotary evaporator. PEG polymers were purchased from Nektar Therapeutics. Fluorescein derivative FAM-SE was purchased from Molecular Probes.

UV-Vis spectroscopic measurements were conducted on a Tidas-II benchtop spectrophotometer (J & M, Germany). Centrifugations were conducted with the following: 1) Allegra 64R Tabletop Centrifuge (Beckman Coulter, Inc., USA); 2) Sorvall RC5C refrigerated high-speed centrifuge (Sorval, USA); or 3) Microfuge® 18 centrifuge) Beckman Coulter, Inc., USA). General desalting and removal of other small molecules of protein samples were achieved using BioSpin® G-25 centrifuge columns (Amersham Biosciences, USA) or NAP-5™ gel filtration columns (Amersham Biosciences, USA). Protein samples were concentrated by way of centrifugal ultrafiltration using Amicon® Ultra-4 or Ultra-15 100 kDa molecular weight cutoff spin columns (Millipore, USA).

¹H NMR and ¹³C NMR spectra were measured with a Bruker AVQ-400 (400 MHz) spectrometer. Chemical shifts are reported as δ in units of parts per million (ppm) relative to dimethyl sulfoxide-d₆ (δ 2.50, pentet). Multiplicities are reported as follows: s (singlet), d (doublet), t (triplet), q (quartet), dd (doublet of doublets), m (multiplet), br (broadened), or app (apparent). Coupling constants are reported as a J value in Hertz (Hz). The number of protons (n) for a given resonance is indicated nH, and is based on spectral integration values. ¹³C NMR spectra are reported as δ in units of parts per million (ppm) relative to dimethyl sulfoxide-d₆ (δ 39.50, septet).

Transmission Electron Microscopy (TEM) images were obtained at the UC-Berkeley Electron Microscope Lab using a FEI Tecnai 12 transmission electron microscope with 100 kV accelerating voltage. Protein samples were prepared for TEM analysis by applying 5 μL of an analyte solution at approximately 0.1 mg/mL to carbon-coated copper grids for 3 min followed by rinsing with ddH₂O. The grids were then exposed to 5 μL of a 1% solution of uranyl acetate (UA) for 1.5 min as a negative stain. After excess stain was removed by blotting, the grid was allowed to dry until analysis.

Matrix Assisted Laser Desorption-Ionization Time of Flight (MALDI-TOF) mass spectra (MS) were obtained using a Voyager-DE from PerSeptive Biosystems. MALDI matrices were prepared daily as saturated solutions (generally 10 mg/mL). For protein and peptide analysis, sinapinic acid, α-cyano-4-hydroxycinnamic acid (CHCA) or 2,4,6-trihydroxyacetophenone (THAP) in 3:2 MeCN:ddH₂O (with 0.1% TFA) were used. In all cases, the spot overlay technique was employed for crystallization. See, e.g., Kussmann et al., J. Mass. Spec. 593 (1997). Intact MS2 capsids were disassembled on the column using reversed-phase HPLC prior to MALDI-MS analysis. General desalting and removal of other small molecules of biological samples were achieved using BioSpin® G-25 centrifuge columns (Amersham Biosciences, USA), μC18 ZipTip® columns (Millipore, USA), NAP-10™ gel filtration columns (Amersham Biosciences USA). Prior to analysis of all MS2 capsid samples, reaction solutions were passed through 50 mg of Sephacryl™ S-300 High Resolution resin (Amersham Biosciences, USA) pre-equilibrated in the desired elution buffer and packed in BioSpin® columns using centrifugation (750 rpm, 2 min, 4° C.). Only assembled MS2 particles elute using this method.

HPLC was performed on an Agilent 1100 Series HPLC System (Agilent Technologies, USA). Small molecule chromatography was achieved on C8 reserved-phase columns with a MeCN:H₂O gradient mobile phase containing 0.1% trifluoroacetic acid. Analytical size exclusion chromatography was accomplished on an Agilent Zorbax® GF-250 with isocratic (0.5 mL/min) flow using an aqueous mobile phase (100 mM Na₂HPO₄ with 0.005% NaN₃, pH 7.3). Sample analysis for all HPLC experiments was achieved with an inline diode array detector (DAD) and an inline fluorescence detector (FLD). Preparative size exclusion chromatography (SEC) was performed on a BioRad® BioLogic™ DuoFlow FPLC System equipped with an S-300 High Resolution Column (Amersham Biosciences, USA) using an aqueous buffer (100 mM Na₂HPO₄ with 0.005% NaN₃, pH 7.3) as the mobile phase.

For protein analysis, sodium dodecyl sulfate-polyacrylamide gel electrophoresis (SDS-PAGE) was accomplished on a Mini-Protean apparatus from Bio-Rad (Hercules, Calif.), following the general protocol of Laemmli. See Laemmli, Nature 227, 680 (1970). Samples were combined 1:1 (v/v) with gel loading buffer containing SDS, DTT, and bromophenol blue and heated at 95° C. for approximately 15 min. After removal of the completed gels from their cassettes, the bottom portions containing unbound dye were excised promptly, after which the gels were submerged in the appropriate solution for rinsing or imaging. Commercially available markers (Bio-Rad) were applied to at least one lane of each gel for calculation of apparent molecular masses. Fluorescence visualization of gels was obtained by UV transillumination at 302 nm. Visualization of protein bands was accomplished by staining with Coomassie® Brilliant Blue R-250. Gel image was performed on an EpiChem3 Darkroom system (UVP, USA). Protein recovery and/or degree of modification was estimated from standard optical density measurements of the observed gel bands with LabWorks™ software (version 4.0.0.8, UVP).

All size, zeta potential, and protein melting curve data were obtaining using a Zetasizer Nano Series (Malvern Instruments Limited, UK). For size and melting curve measurements, analyte solution at approximately 0.5 mg/mL in aqueous buffer was sterile filtered into glass cuvettes equipped with a Teflon cap. For zeta potential measurements, analyte solution at approximately 0.5 mg/mL in 10 mM Na₂HPO₄, pH 7.3 was sterile filtered directly into disposable zeta cells equipped with electrodes and a folded capillary (Malvern Instruments Limited, U.K.).

### Propagation of Bacteriophage MS2

Routine propagation of MS2 was carried out in a one-liter batch process using a modified procedure of Strauss and Sinsheimer (J. Mol. Biol. 43 (1963); Analytical Chemistry 73, 1277 (2001)). The growth medium for the host bacteria, E. coli, was prepared by the addition of 10 g TryptoPeptone, 5 g Bacto™ Yeast Extract, and 8 g NaCl to 1 liter of ddH₂O. After autoclave sterilization of the resulting broth, 10 mL of sterile 10% glucose solution, 2 mL of sterile 1 M CaCl₂ solution, and 1 mL of a sterile 10 mg/mL thiamine hydrochloride solution were added. Culture media were infected with revived Hfr⁺ E. coli that had been grown from a single colony originally isolated from a freeze-dried pellet (American Type Culture Collection, ATCC, No. 15669; Rockville, Md.). The infected culture was incubated at 37° C. under aerobic conditions until the optical density (OD) of 0.2 at 600 nm was reached, signifying exponential growth of the host bacteria. Inoculation of the bacteria was accomplished by the addition of a small aliquot of MS2 suspension stored at 4° C. that had previously been propagated from purchased stock (ATCC No. 15597-B1) by a similar procedure. Propagation of the virus was carried out at 37° C. for at least 4 h, but typically overnight to ensure complete lysis of the bacterial culture. Isolation of the MS2 phage was performed by separation of lysed bacterial debris by centrifugation at 4500 ref for 30 min at 4° C. followed by selective precipitation by the addition of 10% (w/v) poly(ethylene glycol)-6000 and NaCl to a final concentration of 0.5 M. The precipitated MS2 was then separated from the supernatant by centrifugation at 13,000 ref for 1 h at 4° C. The resulting pellet was resuspended in 50 mL of aqueous buffer (0.5 M Na₂HPO₄, 0.1 M NaCl, pH 7.2) and passed through a 0.22 μm sterile filter (Millipore Corp., USA) under vacuum to afford MS2 phage as the only protein in solution, as determined by SDS-PAGE. Further purification of MS2 by FPLC (as described above) was performed to remove residual polymer from the precipitation step.

### Preparation of Genome-Free MS2 Capsids

To a solution of MS2 virions was added 10% (w/v) poly(ethylene glycol)-6000 and NaCl to a final concentration of 0.5 M to precipitate the protein capsids. The precipitate was separated via centrifugation and then dissolved in aqueous buffer (100 mM Na₂HPO₄, 100 mM NaCl, pH 11.8). After 2.5 h at rt, the protein was precipitated as outlined above and redissolved. The precipitate mixture was centrifuged at 10,000 rcf for 30 min at 4° C. and the pellet was redissolved in a minimal volume of aqueous buffer (100 mM Na₂HPO₄, 100 mM NaCl, pH 11.8). After incubation at rt for 1.5 h, the MS2 solution was passed through a gel filtration column via FPLC (as outlined above). The overall process afforded empty capsids in about 80 to about 90% yield of the initial phage.

### Procedure for the Formation of Ketone-Modified MS2 via Conjugation of Lysine Residues Using 1-(4-acetylbenzyl)-1H-pyrrole-2,5-dione

To a vial containing 0.9 mL of a purified solution of intact, native MS2 (1.1 mg/mL) in 0.1 M NaHCO₃ buffer, pH 8.4) was added a solution of 1-(4-acetylbenzyl)-1H-pyrrole-2,5-dione (1) (2.9 μmol) pre-dissolved in 100 μL of dry, distilled DMF. The resulting solution was incubated at room temperature for 3 h with moderate stirring. To separate the modified virus from the small molecules, 10% (w/v) poly(ethylene glycol) with an average molecular weight of 6000 (PEG-6000) along with 0.5 M NaCl was added directly to the crude reaction solution. The mixture was then centrifuged at 9400×g for 1.5 h to selectively precipitate the intact ketone-modified MS2. The supernatant was carefully removed and the pelleted virus was re-dissolved in fresh buffer. The ketone-modified MS2 was characterized by MALDI-TOF MS and size-exclusion chromatography. Typical results indicated complete disappearance of unmodified virus with an average of 3 modifications per viral capsid monomer and a recovery of about 50 to about 70% of the intact viral material.

### Procedure for the Formation of Ketone-Modified MS2 via Conjugation of Cysteine Residues Using 1-(4-acetylbenzyl)-1H-pyrrole-2,5-dione

To a vial containing 0.9 mL of a purified solution of intact, native MS2 (1.1 mg/mL in 0.05 M NaH₂PO₄ buffer, pH 7.4) was added a solution of 1-(4-acetylbenzyl)-1H-pyrrole-2,5-dione (1) (14.6 μmole) pre-dissolved in 100 μL of dry, distilled DMF. The resulting solution was incubated at room temperature for 24 h with moderate stirring. To separate the modified virus from the small molecules, 10% PEG-6000 and 0.5 M NaCl was added directly to the crude reaction solution. The mixture was then centrifuged at 9400×g for 1.5 h to selectively precipitate the intact ketone-modified. MS2. The supernatant was carefully removed and the pelleted virus was re-dissolved in fresh buffer. The ketone-modified MS2 was characterized by MALDI-TOF MS and size-exclusion chromatography. Typical results indicate an average of 1.5 modifications per viral capsid monomer and a recovery of about 80 to about 90% of the intact material.

### General Procedure for Oxime Conjugation of Ketone-Modified MS2 with O-(Methoxypolyethylene glycol)-hydroxylamine

To a vial containing 0.5 mL of a purified solution of ketone-modified MS2 (1.0 mg/mL in 0.05 M NaH₂PO₄ buffer, pH 3.5-6.5) was added O-(methoxypolyethylene glycol)-hydroxylamine (2) (18 μmol). The reaction mixture was further diluted with buffer (0.05 M NaH2PO4, pH 3.5-6.5) to reach a total volume of 1.0 mL. The resulting solution was incubated at room temperature for 24 h with moderate stirring. The reaction mixture was then diluted with buffer and subjected to at least three rounds of centrifugal ultrafiltration through 100 kDa molecular weight cutoff spin columns (Millipore) to remove unreacted O-(methoxypolyethylene glycol)-hydroxylamine. The relative extent of polymer conjugation was monitored via sodium dodecyl sulfate-polyacrylamide gel electrophoresis (SDS-PAGE) following the general protocol of Laemmeli.

### Empty MS2 Capsid Azo-Conjugate (3)

Formation of the Diazonium Salt: To a 10 μL solution of p-nitroanaline (20 mg/mL, 1.45 μmol) was added 5 μL of an aqueous solution of p-toluene sulfonic acid monohydrate (160 mg/mL) at 4° C. The resulting solution was vortexed for 1 min and treated with 5 uL of an aqueous solution of sodium nitrite (32 mg/mL, 2.3 μmol). The solution was briefly vortexed and diazotization was carried out at 4° C. for 1 h to provide the diazonium salt (2). Diazonium Coupling Reaction: To a 1.0 mL aqueous buffered solution (150 mM NaHPO₄) of the MS2 viral capsid (1) containing interior surface tyrosine Y-85 (1.2 mg/mL, 87 nmol, pH 9.0) was added 6.0 μL of the diazonium salt (2) (435 nmol). The resulting solution was vortexed briefly. Diazonium coupling was carried out at 4° C. for 15 min. The reaction solution was passed through a gel filtration column (NAP-10) pre-equilibrated with 5 column volumes of elution buffer (100 mM Na₂HPO₄, pH 7.2) and eluted in 1.5 mL to provide the azo-conjugate (3).

### Empty MS2 Capsid Ortho-Amino-Y85 (4)

To a 1.0 mL solution of the azo-conjugate (3) was added solid Na₂S₂O₄ (20 mg, 85% technical grade). The solution was briefly vortexed and incubated at rt. After 2 h, the reaction solution was passed through a gel filtration column (NAP-10) pre-equilibrated with 5 column volumes of elution buffer (100 mM NaHPO₄, pH 6.5) and eluted in 1.5 mL to provide the ortho-amino-Y85 (4).

### Empty MS2 Capsid Ortho-Amino-Y85 Oxidative Coupling of (4) to Provide (6)

A 100 μL aliquot of 4 (approximately 0.5 mg/mL) was passed through a S-300 microspin gel filtration column (as outlined above) to ensure the removal of dithionite reductant. To the resulting solution was added 1 μL of a given N-acylated-1,4-phenylenediamine (5) in MeCN (100 mM), followed immediately by 1.1 μL of an aqueous solution of sodium periodate (100 mM). The resulting reaction solution was incubated at room temperature for 5 min and then passed through a S-300 microspin gel filtration column to provide (6). Extent of coupling was determined by MALDI-TOF MS. TEM was used to verify that the protein aggregates obtained after SEC were intact capsids.

### MS2-DOTA Conjugate (8)

To a 1.0 mL aqueous buffered solution (100 mM NaHPO₄) of genome-free MS2 capsids (2.0 mg/mL, 145 nmol in MS2 monomer repeat, pH 8.5) was added 10.0 uL of DOTA-NHS (7) (1,4,7,10-tetraazacyclododecane-1,4,7,10-tetraacetic acid mono(N-hydroxysuccinimide ester) PF6⁻ salt) (5 mg/mL in DMF, approximately 7 μmol). The resulting solution was incubated for 4 h at rt on a rotating mixer and then passed through a NAP-10 size exclusion column (eluting in 100 mM NH4OAc buffer, pH 6.0) to provide (8). Extent of coupling was determined by MALDI-TOF MS. SEC was used to verify that the capsids had remained intact.

### MS2-DOTA Metal Chelates (9a-e)

To a 50 uL aqueous buffered solution (100 mM NH₄OAc) of (8) (1.5 mg/mL, approximately 65 nmol in DOTA ligand, pH 6.0) was added 1.0 μL of an aqueous solution of a given metal salt (GdCl₃ or CuCl₂ or Yb(OAc)₃, YbCl₃, EuCl₃, TbCl₃) (1M). The resulting solution was incubated for 2 h at 37° C. on a vortexing mixer and then passed through a S300 size exclusion centrifuge column (eluting in 100 mM NH₄OAc buffer, pH 6.0) to provide any of the metal chelates 9(a)-(e). Extent of metal ion incorporation was determined by MALDI-TOF MS. SEC was used to verify that the capsids had remained intact.

### 5-Amino-N-(2-amino-ethyl)-2-nitrobenzamide (4)

A 200 mL flame dried round bottom flask equipped with a magnetic stir bar was charged with a solution of 5-amino-2-nitrobenzoic acid (750 mg, 4.12 nmol, 1 equiv) in distilled pyridine (25 mL) under an argon atmosphere. To the stirred solution was added 1,1′-carbonyldiimidazole (CDI, 1.00 g, 6.18 mmol, 1.5 eqv) at rt. After 45 min, all of the 5-amino-2-nitrobenzoic acid had been converted to the active imidazole amide at which point the reaction solution was cooled to 0° C. and treated with a solution of ethylene diamine (1.48 g, 24.7 mmol, 6 eqv) in pyridine (25 mL). (Reaction progress was monitored by TLC (1:1 MeOH/CH₂Cl₂). The flask was equipped with a water-jacketed reflux condenser and the reaction solution was heated to 80° C. After 12 h, the solution was cooled to rt and concentrated under reduced pressure. Purification was accomplished by flash chromatography (1:9 CHCl₃:MeOH) affording 4 as a pure yellow solid (767 mg, 83% yield): ¹H NMR (400 MHz, DMSO-d₆): δ 2.60 (t, 2H, J=6.0 Hz), 3.14 (app q, 2H, J=6.0 Hz), 6.43 (d, 1H, J=2.4 Hz), 6.56 (dd, 1H, J=2.4, 9.2 Hz); 6.72 (br s, 2H), 7.89 (d, 1H, J=9.2 Hz), 8.27 (t, 1H, 5.6 Hz). ¹³C NMR (100 MHz, DMSO-d₆): δ 41.6, 43.3, 112.3, 112.7, 127.9, 133.3, 137.7, 155.1, 167.5.

### 5-Amino-N-(2-[FAM-amido]-ethyl)-2-nitrobenzamide (5)

To a microcentrifuge tube was added 4 (10 mg, 45 μmol), FAM-SE (23 mg, 49 μmol), anhydrous triethylamine (12.4 μL, 89 μmol), and anhydrous DMF (77 μL). After thorough vortexing, the resulting dark orange mixture was protected from light and heated at 65° C. for 1.5 h. (Reaction progress was monitored by way of C8 reversed phase HPLC followed by MALDI-TOF MS). After cooling to room temperature, the resulting product mixture was used without further purification. Unused portions were protected from light and stored in aliquots at 4° C.

### FAM-Azo-Labeled mtMS2 Conjugate (2)

a) Formation of Diazonium Salt 6: To a 50 μL solution of 5 in DMF (100 mM) chilled to 4° C. was added 25 μL of a 4° C. aqueous solution of p-toluene sulfonic acid monohydrate (800 mM) followed immediately by 25 μL of a 4° C. aqueous solution of sodium nitrite (200 mM). The resulting bright yellow mixture was thoroughly vortexed and allowed to incubate at 4° C. for 15 min. b) Diazonium Coupling Reaction: To a 12.2 mL aqueous buffered solution of mtMS2 (1.0 mg/mL, 0.89 μmol of Y85, ph 8.5, 100 mM NaHPO₄) at 4° C. was added 89 μL of 6 (4.45 μmol). After brief vortexing, the resulting orange solution was held at 4° C. for 15 min. The reaction mixture was then diluted to 14 mL, after which 1.4 g of poly(ethylene) glycol, average molecular weight 6000 (PEG-6000) and 409 mg of NaCl were added. The resulting heterogeneous mixture was then centrifuges at 13,000×g ref for 0.5 h to precipitate assembled MS2 capsids selectively. Further purification via FPLC (as described above) removed residual polymer and free dye, while final centrifugal ultrafiltration yielded intact, pure, stable FAM-MS2 conjugate 2 with an overall protein recovery of approximately 90% with conversion levels of 50-80 FAM/capsid.

### General Procedure for PEGylation of MS2 Capsids

To 100 μL of an MS2 sample (1 mg/mL, 70 μM in 150 mM NaHCO₃ buffer, pH 8.4) was added 1.82 nmol of PEG-NHS (MW=2000 or 5000). The reaction mixture was vortexed gently for 18 h at room temperature and then subjected to FPLC purification and final centrifugal ultrafiltration as described above.

### Production of Polyclonal Anti-MS2 Antibodies

The production of polyclonal anti-MS2 antibody (Ab) sera was performed as a 90-day accelerated protocol by Covance Research Products (Denver, Pa.). Briefly, two NZW female rabbits, were following a pre-bleed, injected with 250 μg of the purified intact, native MS2 capsid antigen emulsified in Freund's Complete Adjuvant (FCA). Subsequent booster injects of 12 5 μg of antigen in Freund's Incomplete Adjuvant (FIA) were given for each rabbit on days 14, 35, 49 and 70 following the initial injections. Production bleeds were taken on days 59 and 80. Following this, an indirect enzyme-linked immunosorbent assay (ELISA) was taken to measure the antibody titer of each production sample against plates coated with initial antigen. After results indicated acceptable antibody titers, a terminal bleed for each rabbit was performed, yielding 60 mL of serum per rabbit. Serum samples for immediate use in sandwich ELISA experiments were stored for several months at 2-8° C. Aliquots of the remaining sera were frozen and stored at −20° C.

### Sandwich ELISA Assay

Affinity-purified polyclonal anti-MS2 Abs were dilutes 1000-fold into 50 mM NaHCO₃ buffer, pH 8.2, added (50 μL/well) to Reaction-Bind 96-Well EIA Plates (Pierce Endogen, USA) and incubated overnight at 4° C. The plates were then washed once with phosphate buffered saline containing 0.1% Tween-20 and 0.05% NaN₃ (PBS/Tween/azide) followed by a single wash with PBS/azide before being blocked with 2% bovine serum albumin (BSA) in PBS/Tween/azide. After a two hour incubation at room temperature, the plates were washed three times with PBS/Tween/azide, once with PBS/azide, and dried briefly. To each well was next added 50 μL of the appropriate MS2 or control protein sample at a concentration of 10-100,000 ng/mL, diluted in 2% BSA in PBS/Tween/azide. After a two hour incubation at room temperature, the plates were washed as before. A previously prepared polyclonal anti-MS2 Ab-horseradish peroxidase (Ab-HRP) conjugate was diluted 100-fold in PBS/Tween (no azide) and added (50 μL/well) to the plates, which were then incubated at room temperature for two hours. The plates were washed three times with PBS/Tween and once with PBS before addition (100 μL/well) of 1-Step ABTS substrate solution™ (Pierce Endogen). Following a 15-30 min incubation at room temperature, each well was quenched with 100 μL of a 1% aqueous solution of SDS. Absorbance readings were measured at 405 and 410 nm on a 96-well plate reader.

### Results

### MS2 as an Exemplary Target Capsid Scaffold

A exemplary target scaffold, bacteriophage MS2, possesses many desirable features with regard to both processing and synthesis. It is a T=3 icosahedral virus with a 27 nm protein capsid composed of 180 identical copies of a 13.7 kDa subunit. See, e.g., Valegard et al., Nature 345, 36-41 (1990) and Golmohammadi et al. J. Mol. Biol. 234, 620-639 (1993). Native MS2 (natMS2) is safe, easily handled, readily propagated in multi-milligram quantities in broth cultures and remains indefinitely stable upon storage in aqueous buffer at 4° C. See, e.g., Davis et al., J. Mol. Biol. 6, 203-207 (1963) and Cargile et al., Anal. Chem. 73, 1277-1285 (2001).

In addition, we have previously shown that natMS2 possesses impressive stability to a broad range of temperature, pH, ionic strength, and organic co-solvent conditions, and it is capable of forming genome-free “empty” capsid shells (mtMS2, 1) that exhibits nearly identical resistance to disassembly and denaturation under extreme conditions. See, e.g., Hooker et al., J. Am. Chem. Soc. 126, 3718-3719 (2004). Crucial to our internal modifications is the fact that MS2 possess 32 identical pores per capsid, each approximately 1.8 nm in diameter. These pores provide ready access to its interior space for moderately sized particles and reagents, including functionalized drug molecules, for covalent attachment.

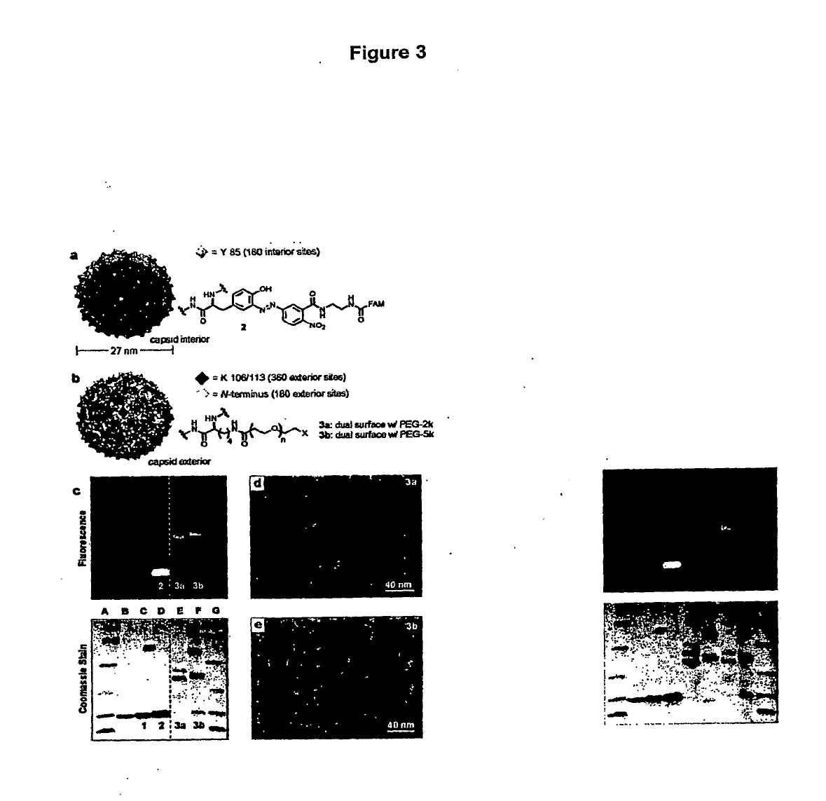

The abundance of reactive amines (the coat protein N-terminus, Lys 106, and Lys 113) presented on the nat- or mt-MS2 exterior allows for facile and high yielding modification via N-hydroxysuccinimides, FIG. 3b. However, the key to selectivity for our dual surface modification strategy is the targeting of an interior amino acid side chain with orthogonal reactivity to lysine and cysteine residues. We have previously shown that this can be accomplished by functionalizing Tyr 85, which is displayed on the interior face of each monomer after capsid assembly. After removal of the RNA genome, this residue can undergo a rapid and efficient coupling to the diazonium salt of p-nitroaniline to install an azo linkage ortho to the phenolic moiety of the tyrosine, FIG. 3a. See, e.g., Hooker et al., J. Am. Chem. Soc. 126, 3718-3719 (2004).

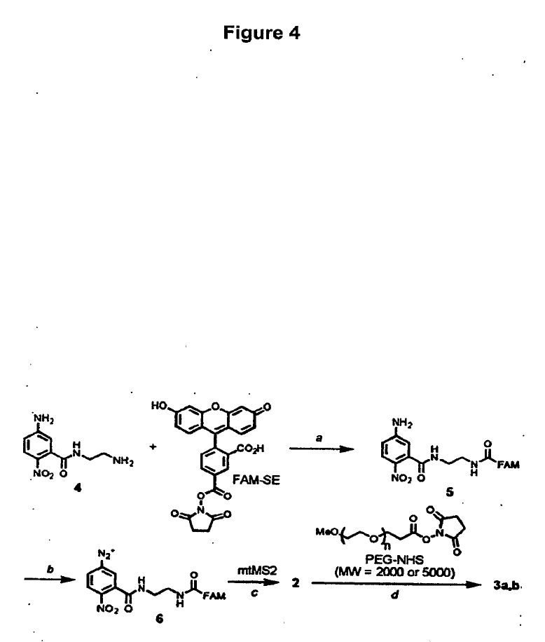

As an elaboration of this strategy, the current study began with the synthesis of aniline 4 of FIG. 4, which bears the nitro group that is required to achieve high coupling efficiency and an aliphatic amino group that can be functionalized selectively with virtually any commercially available NHS-ester. This is demonstrated for fluorescein derivative FAM-SE in FIG. 4. The resulting aniline derivative 5 of FIG. 4 was then converted to diazonium salt 6 of FIG. 4 before being exposed to mtMS2 in a 5 molar excess (relative to capsid monomers) for 15 min. After gel filtration to remove small molecules and any dissociated monomers, the resulting conjugate 2 of FIG. 4 was recovered at 90% with each intact particle encapsidating 50-70 fluorescein dyes (as determined by MALDI-TOF MS and subsequently corroborated by UV-Vis). We were particularly interested in these fluorescent anilines, as they can serve as a useful visualization probe for subsequent cell-based assays. In addition, the large size of the aniline-dye conjugate (MW of approximately 600) demonstrates that access to the capsid interior is permitted for relatively large molecules, including many drugs. With the internally labeled capsids in hand, the exterior surface was passivated through the attachment of poly(ethylene) glycol (PEG) chains. See, e.g., Bailon et al., Pharm. Sci. Technol. Today 1, 352-356 (1998) and Zaplinsky et al., Chemistry and Biological Applications of Polyethylene Glycol 680, 1-15 (1997).

As previously demonstrated for both viral capsids and numerous other substrates, PEG conjugation reduces the humoral immune response in vivo by masking the epitopes of the parent scaffold. See, e.g., O'Riordan et al., Hum. Gene Ther. 10, 1349-1358 (1999); Croyle et al., 11, 1713-1722 (2000); Raja at al., Biomacromolecules 4, 472-476 (2003); Wang et al., Bioconj. Chem. 14, 38-43 (2003); Harris et al., Nature Rev. Drug Discov. 2, 214-221 (2003); Duncan, Nature Rev. Drug Discov. 2, 347-360 (2003). Along with reduced immunogenicity, PEGylation of a vast array of biomacromolecules and small molecules has been shown to improve serum half-life, enhance bioavailability and minimize proteolytic cleavage. For initial studies, the attachment of PEG chains was accomplished through the reaction of 2 with commercially available PEG-NHS esters (MS=2000 or 5000), affording conjugates 3a and 3b with essentially complete conversion (see FIG. 4, step d). Gel filtration using S-300 resin, followed by centrifugal concentration, removed the excess PEG after bioconjugation was complete. For both polymers, single, doubly, and triply modified capsid monomers were observed by SDS-PAGE. In comparison to other viral capsid-based scaffolds, this level of PEG coverage is unprecedented, adding over 1800 kD of polymer (in the case of the 5k-PEG) to the surface of each capsid. This coverage, furthermore, is obtained without any observable protein precipitation or denaturation, in contrast to what has been observed previously for other viral particles. Fluorescence imaging of the gel for conjugates 3a and 3b confirmed that the polymer-modified monomers retained the dye molecules attached to the inner surface. The selectively differentiated conjugates were obtained with an overall protein recovery.(two steps) of over 80%.

The assembly state of the doubly modified capsids was determined using several analytical techniques. Analytical size exclusion chromatography (see Supporting Information) and TEM analyses (FIGS. 3d, 3e) indicated the presence of intact capsids with no detectable presence of protein monomers. In addition, the spherical nature of the capsid conjugates made them particularly amenable to further characterization by dynamic light scattering (DLS). See, e.g., Table 1. Virtually identical particle diameters were observed for natMS2, mtMS2, and 2, each measuring 27-28 nm. Upon conjugation of the PEG-2k chains 3a, an effective diameter of 34 nm was observed, with a further increase to 41 nm upon conjugation of the PEG-5k chains 3b. Further determination of each particle's zeta potential indicated that the inherent excess negative surface charge of MS2 (−19.40 mV) is reduced significantly upon PEGylation, a property that is highly desirable to avoid bioaccumulation in drug delivery formulations. Interestingly, thermal denaturation studies (also monitored using DLS) indicated that the dual surface-functionalized particles remained fully assembled up to 58° C., with no apparent effects from the polymer coating. This value is identical to that measured for natMS2 itself.

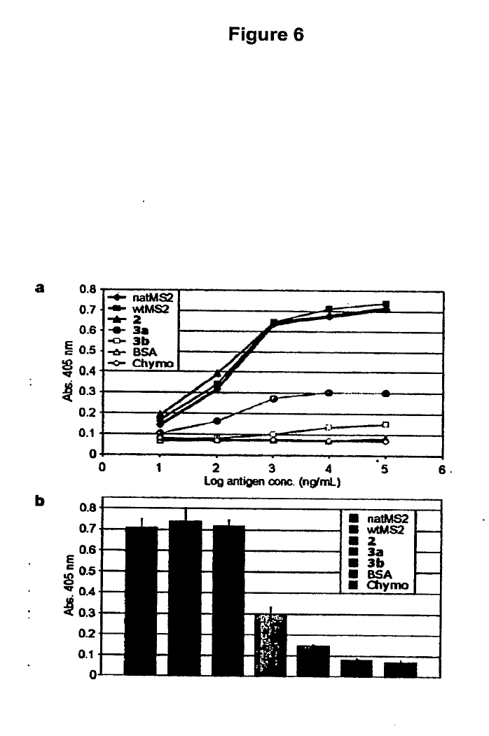

To determine the ability of the PEG chains to mask the capsid surface from antibody recognition, we subjected our samples to a sandwich ELISA assay. Using rabbit-derived anti-native MS2 polyclonal antibodies for detection and capture, identical maximum antibody binding was achieved with either natMS2, mtMS2, or conjugate 2 under the full range of protein concentrations tested. This indicates that both genome removal and subsequent internal modification with 6 fails to mask the native epitopes of the MS2 capsid. See FIG. 6. In contrast, PEG-2000 conjugate 3a exhibited greater than 65% reduction in antibody binding, while PEG-5000 conjugate 3b showed nearly 90% reduction at the highest concentration of antigen sample tested, FIG. 6b. These results demonstrate that our extensive external PEGylation significantly shields the MS2 capsid from antibody binding in vitro.

A number of functional groups have been shown to direct attached cargo to specific tissue types in vivo. In terms of small molecules, folate, biotin, and vitamin B₁₂ have been particularly successful for the targeting of solid tumors and a growing collection of peptides targeting organs of interest have been identified through the use of phage display techniques. In addition to shielding the capsid surface from antibody recognition, it was envisioned that the polymer could be used to display these molecules to gain similar control of cellular uptake for MS2-derived drug carriers. However, a key consideration of this technique is the ability of the small molecules to reach their targets in the presence of the PEG layer. To test this, samples of mtMS2 capsids were biotinylated using commercially available NHS esters bearing (in the case of 8a and 8b) or lacking (in the case of 7) PEG chains. For biotin-3k-PEG conjugate 8a, SDS PAGE analysis indicated that 30% of the monomers has been functionalized, while for biotin-5k-PEG conjugate 8b, the level of modification was 70%. These differences in reactivity were attributed to degradation of the NHS esters during storage prior to use. Nonetheless, this method produced samples displaying 50-100 biotin groups at the end of the polymer chains.

The resulting samples were exposed to avidin beads for 1 and 15 h, after which the samples were centrifuges to remove the bound MS2 from solution. SDS-PAGE analysis of the remaining supernatants revealed 83% recovery for mtMS2 capsids as a negative control. Surprisingly, similar levels (87%) of mtMS2 capsids functionalized with biotin were also recovered, suggesting that the capsid surface hinders the ability of the avidin groups to reach the displayed ligands. In contrast, both of the PEG-biotin conjugates were removed from solution using this method, indicating that the small molecule ligands had some access to the solution surrounding the PEG layer. After 15 h of exposure to the resin, 90% of the capsids were removed from solution for both 8a and 8b. It should be noted that these effects will vary dramatically for each ligand-receptor combination; however, this experiment validates the placement of targeting groups at the ends of the polymer chains.

Following the success of this experiment, the final synthetic component of these studies was the development of a modular strategy for the coupling of a wide variety of small molecules to the ends of the PEG chains. To do this, oxime formation chemistry was chosen, as it proceeds rapidly and chemoselectively under mild reaction conditions in aqueous media. In a previous report, we have explored the use of PEG-alkoxyamines, such as methoxy-terminated 11, for the modification of ketones introduced on the surface of the tobacco mosaic virus. For this study, precursors to PEG-bis-alkoxyamines were prepared through the displacement of the alcohol groups of PEG-diol 10 using hydroxyphthalamides under Mitsunobu conditions. Hydrazinolysis subsequently afforded bis-alkoxyamine 12, which could be condensed with ketones and aldehydes in CH₂Cl₂ using catalytic TFA. Fluorescent coumarin-ketone 13 was used in these model studies, as it can be visualized after SDS-PAGE analysis of the bioconjugates.

To bind to the remaining alkoxyamine group of 14, aldehydes were installed on the surface of mtMS2 capsids using NHS ester 15. Following removal of the small molecules using gel filtration, MALDI-MS analysis indicated a general conversion of the capsid monomers to single, double, and triple conjugates. These groups were readily attached to the alkoxyamine groups in aqueous phosphate buffer, pH 6.5, in 1.5 h at rt. The unreacted PEG chains were removed using gel filtration, and the samples were analyzed using SDS-PAGE with fluorescence visualization of the attached coumarin, followed by Coomassie staining. Using these methods, high levels of conversion could be obtained to the single, double, and triply-conjugated species. It was also observed that the number of ligands that are displayed could be modulated by varying the ratio of labeled PEG 17 to methoxy-PEG 11.

In these studies, we have demonstrated the ability of MS2 viral capsids to undergo a facile, site-selective dual surface modification to afford unique hybrid drug delivery vehicles. These conjugations were achieved in very high overall yield, were accomplished without the need for site-directed mutagenesis, and produced particles as impressively resistant to disassembly and denaturation as the native capsid. Our exterior PEGylation levels match or exceed previous reports for other viral particles, while avoiding the undesired precipitation that often accompanies these conjugations. This extensive coverage, further, results in a nearly 90% reduction in antibody binding of the capsid surface. The interior modification strategy demonstrates that access to the interior is readily permitted for large “drug-like” dye molecules while further expanding the utility of tyrosine bioconjugations to generate high-yielding, unique-selective, and stable constructs.

It is understood that the examples and embodiments described herein are for illustrative purposes only and that various modifications or changes in light thereof will be suggested to persons skilled in the art and are to be included within the spirit and purview of this application and scope of the appended claims. All publications, patents and patent applications cited herein are hereby incorporated by reference in their entirety for all purposes.

### REFERENCES

1. Wang et al., Chem. Biol. 2002, 9, 813-819
2. Klem et al., J. Am. Chem. Soc. 2003, 125, 10806-10807
3. Allen et al., Magnetic Resonance in Medicine 2005, 54, 807-812
4. Gupta et al., Bioconj. Chem. 2005, 16, 1572-1579
5. Wang et al. Chem. Biol. 2002, 9, 805-811
6. Wang et al. Angew. Chem. Int. Ed. 2002, 41, 459-462
7. Gillitzer et al. Chem. Commun. 2002, 2390-2391
8. Schlick et al. J. Am. Chem. Soc. 2005, 127, 3718-3723
9. Raja et al. Chembio Chem 2003, 4, 1348-1351
10. Gupta et al. Chem Commun. 2005, 4315-4317
11. Strable et al. Nano Lett. 2004, 4, 1385-1389
12. Wang et al. J. Am. Chem. Soc. 2003, 125, 3192-3193
13. Douglas et al. Nature 1998, 393, 152-155
14. Douglas et al. Adv. Mater. 1999, 11, 679-681
15. Douglas et al. Adv. Mater. 1999, 14, 415-418
16. Loo et al. J. Am. Chem. Soc. 2006, 128, 4502-4503
17. Mastico et al. J. Gen. Virol. 1993, 74, 541-548
18. Burke et al. Nature, 1988, 332, 81-82
19. Heal et al. Vaccine 1999, 18, 251-258
20. van Meerten et al. J. Gen. Virol. 2001, 82, 1797-1805
21. Chatterji et al. Intervirology 2002, 45, 362-370
22. Zwick et al. Curr. Opinion Biotechnol. 1998, 9, 427-436
23. Whaley et al. Nature 2000, 405, 665-668
24. Lee et al. Science 2002, 296, 892-895
25. Henry, Chem. Eng. News 2002, 80(34), 39-47
26. Matsumura et al. Cancer Res. 1986, 6, 6387-6392
27. Seymour et al. Brit. J. Cancer 1994, 70, 636-641.
28. Gianasi et al. Eur. J. Cancer 1999, 35, 994-1002
29. Strauss et al. Analytical Chemistry 2001, 73, 1277

TABLE 1
Properties of MS2 conjugates obtained from DLS measurements.

| Sample | Avg. particle size^{a} | Zeta Potential^{b} | Disassembly Temp^{c} |
|---|---|---|---|
| mtMS2 | 28.9 ± 1.1 nm | −19.4 ± 2.3 mV | 58-61° C. |
| 2 | 27.5 ± 0.9 nm | −21.5 ± 1.4 mV | 58-61° C. |
| 3a | 34.5 ± 0.7 nm | −11.4 ± 0.6 mV | 58-61° C. |
| 3b | 40.9 ± 1.0 nm | −5.0 ± 2.1 mV | 58-61° C. |

^{a}Average of ten separate measurements.
^{b}Average of three separate measurements.
^{c}Reported as the point at which additional peaks (±50%; of established particle size) emerge as >4% of the volume distribution for a single particle size measurement.

What is claimed is:

1. A method of using a viral capsid comprising an interior surface and an exterior surface to deliver a therapeutic agent to a selected cell of a mammal in need thereof, comprising
modifying the exterior surface by covalently attaching a polymer,
removing the capsid's native genome, and
modifying the interior surface by covalently attaching a therapeutic agent, wherein the covalent attachment is cleaved by conditions present in the cell, and
administering the resulting modified capsid to the mammal.

2. The method according to claim 1, wherein the native genome is RNA.

3. The method according to claim 1, wherein the modifying of the interior surface comprises
treating an amino acid residue that is affixed to the interior surface of the capsid with the therapeutic agent under conditions sufficient to covalently attach the therapeutic agent directly or indirectly to the amino acid residue.

4. The method according to claim 3, wherein the amino acid residue contains a thiol or alcohol or amino moiety that is directly or indirect attached covalently to the therapeutic agent.

5. The method according to claim 3, wherein the amino acid residue is selected from the group consisting of cysteine, serine, tyrosine, lysine and arginine.

6. The method according to claim 5, wherein the amino acid residue is a tyrosine.

7. The method according to claim 6, wherein at least 60 percent of the available tyrosines on the interior surface have been modified.

8. The method according to claim 7, wherein at least 80 percent of the available tyrosines on the interior surface have been modified.

9. The method according to claim 8, wherein at least 95 percent of the available tyrosines on the interior surface have been modified.

10. The method according to claim 1, wherein the cleavage of the covalent attachment occurs under acidic pH conditions present in the cell.

11. The method according to claim 10, wherein the pH is between about 4 to about 6.

12. The method according to claim 11, wherein the pH is about 4.5.

13. The method according to claim 11, wherein the pH is about 5.5.

14. The method according to claim 1, wherein the therapeutic agent is an anti-cancer agent.

15. The method according to claim 1, wherein the modifying of the exterior surface comprises
treating an amino acid residue that is affixed to the exterior surface of the capsid with the polymer under conditions sufficient to covalently attach the polymer directly or indirectly to the amino acid residue.

16. The method according to claim 15, wherein the amino acid residue contains a thiol or alcohol or amino moiety that is directly or indirectly attached covalently to the polymer.

17. The method according to claim 16, wherein the amino acid residue is selected from the group consisting of cysteine, serine, tyrosine, lysine and arginine.

18. The method according to claim 16, wherein the amino acid residue is a cysteine.

19. The method according to claim 15, wherein the amino acid residue is attached to the polymer through a linking group.

20. The method according to claim 1, wherein the polymer is a polyalkoxyamine.

21. The method according to claim 20, wherein the polyalkoxyamine is a poly(ethylene glycol)alkoxyamine.

22. The method according to claim 21, wherein the poly(ethylene glycol) alkoxyamine is an O-(methoxypolyethylene glycol)alkoxyamine.

23. The method according to claim 21, wherein the poly(ethylene glycol) portion of a single chain of the poly(ethylene glycol)alkoxyamine has a MW of about 1,000 to about 25,000.

24. The method according claim 23, wherein the poly(ethylene glycol) portion has a MW of about 2,000.

25. The method according to claim 23, wherein the poly(ethylene glycol) portion has a MW of about 5,000.

26. The method according to claim 1, wherein the viral capsid is a MS2 Bacteriophage.

27. The method according to claim 24, wherein the viral capsid is a MS2 Bacteriophage and the total amount of the poly(ethylene glycol) portion of the poly(ethylene glycol)alkoxyamine added to the exterior of the capsid is about 650,000 kDa.

28. The method according to claim 25, wherein the viral capsid is a MS2 Bacteriophage and the total amount of the poly(ethylene glycol) portion of the poly(ethylene glycol)alkoxyamine added to the exterior of the capsid is about 1.5 MDa.

29. A method of using a viral capsid comprising an interior surface and an exterior surface to deliver a diagnostic imaging agent to a selected cell of a mammal in need thereof, comprising
removing the capsid's native genome,
modifying at least one of the exterior surface and the interior surface by covalently attaching the diagnostic imaging agent, and
administering the resulting modified capsid to the mammal.

30. The method according to claim 29, wherein the modifying of the at least one of the exterior surface and the interior surface comprises
treating an amino acid residue that is affixed to the at least one surface of the capsid with the diagnostic imaging agent under conditions sufficient to covalently attach the diagnostic imaging agent directly or indirectly to the amino acid residue.

31. The method according to claim 30, wherein the amino acid residue contains a thiol or alcohol or amino moiety that participates in the covalent attachment to the diagnostic agent.

32. The method according to claim 31, wherein the amino acid residue is selected from the group consisting of cysteine, serine, tyrosine, lysine and arginine.

33. The method according to claim 31, wherein the amino acid residue is a lysine.

34. The method according to claim 30, wherein the at least one of the exterior surface and the interior surface is the exterior surface.

35. The method according to claim 30, wherein the amino acid residue is attached to the diagnostic imaging agent through a linking group.

36. The method according to claim 29, wherein the diagnostic imaging agent is a complex formed by combining a metal-binding ligand with a metal ion.

37. The method according to claim 35, wherein the metal ion is selected from the group consisting of Gd(III), Cu(II), Tb(III), Yb(III) and Eu(III).

38. The method according to claim 36, wherein the metal ion is Eu(III).

39. The method according to claim 29, wherein the diagnostic imaging agent is a radionuclide.

40. The method according to claim 39, wherein the radionuclide is ¹⁸F or ¹³¹I.

41. The method according to claim 34, further comprising modifying the exterior surface by covalently attaching a polymer.

42. The method according to claim 41, wherein the modifying of the exterior surface comprises
treating an amino acid residue that is affixed to the exterior surface of the capsid with the polymer under conditions sufficient to covalently attach the polymer directly or indirectly to the amino acid residue.

43. The method according to claim 42, wherein the amino acid residue is a lysine.

44. The method according to claim 42, wherein the polymer is a polyalkoxyamine.

45. The method according to claim 44, wherein the polyalkoxyamine is a poly(ethylene glycol)alkoxyamine.

46. The method according to claim 45, wherein the poly(ethylene glycol) alkoxyamine is an O-(methoxypolyethylene glycol)alkoxyamine.

47. The method according to claim 30, wherein the at least one of the exterior surface and the interior surface is the interior surface.

48. The method according to claim 47, wherein the amino acid residue is a tyrosine.

49. A viral capsid for delivery of a therapeutic agent to selected cells of a mammal in need thereof, comprising
an interior surface to which a therapeutic agent is covalently attached; and
an exterior surface to which a polymer is covalently attached, wherein the capsid's native genome has been removed.

50. The viral capsid according to claim 49, wherein the therapeutic agent is covalently attached to the interior surface of the capsid directly or indirectly through an amino acid residue.

51. The viral capsid according to claim 50, wherein the amino acid residue is a tyrosine.

52. The viral capsid according to claim 51, wherein at least 60 percent of the available tyrosines on the interior surface have been modified.

53. The viral capsid according to claim 51, wherein at least 80 percent of the available tyrosines on the interior surface have been modified.

54. The viral capsid according to claim 51, wherein at least 95 percent of the available tyrosines on the interior surface have been modified.

55. The viral capsid according to claim 49, wherein the therapeutic agent is an anticancer agent.

56. The viral capsid according to claim 49, wherein the polymer is covalently attached to the exterior surface through an amino acid residue.

57. The viral capsid according to claim 56, wherein the polymer is covalently attached to the amino acid residue through a linking group.

58. The viral capsid according to claim 56, wherein the amino acid residue is a cysteine.

59. The viral capsid according to claim 49, wherein the polymer is a polyalkoxyamine.

60. The viral capsid according to claim 59, wherein the polyalkoxyamine is a poly(ethylene glycol)alkoxyamine.

61. The viral capsid according to claim 60, wherein the poly(ethylene glycol) alkoxyamine is an O-(methoxypolyethylene glycol)alkoxyamine.

62. The viral capsid according to claim 61, wherein the poly(ethylene glycol) portion of a single chain of the poly(ethylene glycol)alkoxyamine has a MW of about 1,000 to about 25,000.

63. The viral capsid according claim 62, wherein the poly(ethylene glycol) portion has a MW of about 2,000.

64. The viral capsid according to claim 62, wherein the poly(ethylene glycol) portion has a MW of about 5,000.

65. The viral capsid according to claim 49, wherein the viral capsid is a MS2 Bacteriophage.

66. The viral capsid according to claim 63, wherein the viral capsid is a MS2 Bacteriophage and the total amount of the poly(ethylene glycol) portion of the poly(ethylene glycol)alkoxyamine added to the exterior of the capsid is about 650,000 kDa.

67. The method according to claim 64, wherein the viral capsid is a MS2 Bacteriophage and the total amount of the poly(ethylene glycol) portion of the poly(ethylene glycol)alkoxyamine added to the exterior of the capsid is about 1.5 MDa.

68. A viral capsid for delivery of a therapeutic agent to selected cells of a mammal in need thereof, comprising
an exterior surface to which a diagnostic imaging agent is covalently attached, wherein the capsid's native genome has been removed.

69. The viral capsid according to claim 68, wherein the diagnostic imaging agent is covalently attached to the exterior surface of the capsid directly or indirectly through an amino acid residue.

70. The viral capsid according to claim 68, wherein the diagnostic imaging agent is covalently attached to the amino acid residue through a linking group.

71. The viral capsid according to claim 69, wherein the amino acid residue is a lysine.

72. The viral capsid according to claim 69, wherein the diagnostic imaging agent is a complex formed by combining a metal-binding ligand with a metal ion.

73. The viral capsid according to claim 72, wherein the metal ion is selected from the group consisting of Gd(III), Cu(II), Tb(III), Yb(III) and Eu(III).

74. The viral capsid according to claim 73, wherein the metal ion is Eu(III).

75. The method according to claim 69, wherein the diagnostic imaging agent is a radionuclide.

76. The method according to claim 75, wherein the radionuclide is ¹⁸F or ¹³¹I.

77. The viral capsid according to claim 68, further comprising a polymer covalently attached to the exterior surface.

78. The viral capsid according to claim 77, wherein the polymer is covalently attached to the exterior surface of the capsid directly or indirectly through an amino acid residue.

79. The viral capsid according to claim 78, wherein the polymer is covalently attached to the amino acid residue through a linking group.

80. The viral capsid according to claim 78, wherein the amino acid residue is a lysine.

81. The viral capsid according to claim 77, wherein the polymer is a polyalkoxyamine.

82. The viral capsid according to claim 81, wherein the polyalkoxyamine is a poly(ethylene glycol)alkoxyamine.

83. The viral capsid according to claim 82, wherein the poly(ethylene glycol) alkoxyamine is an O-(methoxypolyethylene glycol)alkoxyamine.

84. A viral capsid for delivery of a therapeutic agent to selected cells of a mammal in need thereof, comprising
an interior surface to which a diagnostic imaging agent is covalently attached, wherein the capsid's native genome has been removed.

85. The viral capsid according to claim 84, wherein the diagnostic imaging agent is covalently attached to the interior surface of the capsid directly or indirectly through an amino acid residue.

86. The viral capsid according to claim 85, wherein the amino acid residue is a tyrosine.
